SEQUENCE LISTING (45 sequences)

SEQ ID NO: 1 - protein, 120 aa, Artificial sequence
  Synthetic amino acid
QVQLQQPGAELVKPGASVKLSCKASGYTFASYWMQWVKQRPGQGLEWIGEIDPSDSYTNYNQKFKGKATLTVDTSSNTAYMQLSSLTSEDSAVYYCARNGNYYGSTYDYWGQGTTLTVSS

SEQ ID NO: 2 - protein, 108 aa, Artificial sequence
  Synthetic amino acid
DIQMTQTTSSLSASLGDRVTISCRASQDVSNYLNWYQQKPDGSVKLLIYYTSRLHSGVPSRFSGSGSGAHYSLTISNLEQEDIATYFCQQGNTLPPWTFGGGTKLEIK

SEQ ID NO: 3 - DNA, 360 bp, Artificial sequence
  Synthetic oligonucleotide
caggtccaactgcagcagcctggggctgagcttgtgaagcctggggcttcagtgaagctgtcctgcaaggcttctggctacaccttcgccagctactggatgcagtgggtaaaacagaggcctggacagggccttgagtggatcggagagattgatccttctgatagctatactaactacaatcaaaagttcaagggcaaggccacattgactgtagacacatcctccaacacagcctacatgcagctcagcagcctgacatctgaggactctgcggtctattactgtgcaagaaatgggaattactacggtagtacctacgactactggggccaaggcaccactctcacagtctcctca

SEQ ID NO: 4 - DNA, 324 bp, Artificial sequence
  Synthetic oligonucleotide
gatatccagatgacacagactacatcctccctgtctgcctctctgggagacagagtcaccatcagttgcagggcaagtcaggacgttagcaattatttaaactggtatcagcagaaaccagatggatctgttaaactcctgatctactacacttcaagattacactcaggagtcccatcaaggttcagtggcagtgggtctggagcacattattctctcaccattagcaacctggagcaagaagatattgccacttacttttgccaacagggtaatacgcttcctccgtggacgttcggtggaggcaccaagctggaaatcaag

SEQ ID NO: 5 - protein, 5 aa, Artificial sequence
  Synthetic amino acid
SYWMQ

SEQ ID NO: 6 - protein, 17 aa, Artificial sequence
  Synthetic amino acid
EIDPSDSYTNYNQKFKG

SEQ ID NO: 7 - protein, 11 aa, Artificial sequence
  Synthetic amino acid
NGNYYGSTYDY

SEQ ID NO: 8 - protein, 11 aa, Artificial sequence
  Synthetic amino acid
RASQDVSNYLN

SEQ ID NO: 9 - protein, 7 aa, Artificial sequence
  Synthetic amino acid
YTSRLHS

SEQ ID NO: 10 - protein, 10 aa, Artificial sequence
  Synthetic amino acid
QQGNTLPPWT

SEQ ID NO: 11 - protein, 19 aa, Artificial sequence
  Synthetic amino acid
MGWSCIILFLVATATGVHS

SEQ ID NO: 12 - protein, 15 aa, Artificial sequence
  Synthetic amino acid
GGGGSGGGGSGGGGS

SEQ ID NO: 13 - protein, 232 aa, Artificial sequence
  Synthetic amino acid
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 14 - protein, 22 aa, Artificial sequence
  Synthetic amino acid
GSGATNFSLLKQAGDVEENPGP

SEQ ID NO: 15 - protein, 44 aa, Artificial sequence
  Synthetic amino acid
SIATGMVGALLLLLVVALGIGLFMRRRHIVRKRTLRRLLQEREL

SEQ ID NO: 16 - DNA, 62 bp, Artificial sequence
  Synthetic oligonucleotide
ccaccatgggatggagctgtatcatcctcttcttggtagcaacagctacaggtgtccactct

SEQ ID NO: 17 - DNA, 45 bp, Artificial sequence
  Synthetic oligonucleotide
gggggcggcggatccgggggagggggttctggcggaggtgggtcc

SEQ ID NO: 18 - DNA, 696 bp, Artificial sequence
  Synthetic oligonucleotide
cccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 19 - DNA, 63 bp, Artificial sequence
  Synthetic oligonucleotide
gggagcggagcgacgaatttcagcctgctgaaacaggctggagatgtggaggagaacccgggc

SEQ ID NO: 20 - DNA, 135 bp, Artificial sequence
  Synthetic oligonucleotide
tccatcgccactgggatggtgggggccctcctcttgctgctggtggtggccctggggatcggcctcttcatgcgaaggcgccacatcgttcggaagcgcacgctgcggaggctgctgcaggagagggagctttga

SEQ ID NO: 21 - protein, 5 aa, Artificial sequence
  Synthetic peptide
GGGGS

SEQ ID NO: 22 - protein, 8 aa, Artificial sequence
  Synthetic peptide
GGGGGGGG

SEQ ID NO: 23 - protein, 6 aa, Artificial sequence
  Synthetic peptide
GGGGGG

SEQ ID NO: 24 - protein, 15 aa, Artificial sequence
  Synthetic peptide
EAAAKEAAAKEAAAK

SEQ ID NO: 25 - protein, 5 aa, Artificial sequence
  Synthetic peptide
EAAAK

SEQ ID NO: 26 - protein, 46 aa, Artificial sequence
  Synthetic peptide
AEAAAKEAAAKEAAAKEAAAKALEAEAAAKEAAAKEAAAKEAAAKA

SEQ ID NO: 27 - protein, 5 aa, Artificial sequence
  Synthetic peptide
PAPAP

SEQ ID NO: 28 - protein, 12 aa, Artificial sequence
  Synthetic peptide
AEAAAKEAAAKA

SEQ ID NO: 29 - protein, 17 aa, Artificial sequence
  Synthetic peptide
VSQTSKLTRAETVFPDV

SEQ ID NO: 30 - protein, 6 aa, Artificial sequence
  Synthetic peptide
PLGLWA

SEQ ID NO: 31 - protein, 6 aa, Artificial sequence
  Synthetic peptide
RVLAEA

SEQ ID NO: 32 - protein, 10 aa, Artificial sequence
  Synthetic peptide
EDVVCCSMSY

SEQ ID NO: 33 - protein, 8 aa, Artificial sequence
  Synthetic peptide
GGIEGRGS

SEQ ID NO: 34 - protein, 10 aa, Artificial sequence
  Synthetic peptide
TRHRQPRGWE

SEQ ID NO: 35 - protein, 10 aa, Artificial sequence
  Synthetic peptide
AGNRVRRSVG

SEQ ID NO: 36 - protein, 9 aa, Artificial sequence
  Synthetic peptide
RRRRRRRRR

SEQ ID NO: 37 - protein, 4 aa, Artificial sequence
  Synthetic peptide
GFLG

SEQ ID NO: 38 - protein, 18 aa, Artificial sequence
  Synthetic peptide
KESGSVSSEQLAQFRSLD

SEQ ID NO: 39 - protein, 14 aa, Artificial sequence
  Synthetic peptide
EGKSSGSGSESKST

SEQ ID NO: 40 - protein, 12 aa, Artificial sequence
  Synthetic peptide
GSAGSAAGSGEF

SEQ ID NO: 41 - protein, 19 aa, Artificial sequence
  Synthetic peptide
MGKWVKVLFALICIAVAES

SEQ ID NO: 42 - protein, 16 aa, Artificial sequence
  Synthetic peptide
METPAQLLFLLLLWLP

SEQ ID NO: 43 - protein, 16 aa, Artificial sequence
  Synthetic peptide
MGWSCIILFLVATATG

SEQ ID NO: 44 - protein, 20 aa, Artificial sequence
  Synthetic peptide
MSVPTQVLGLLLLWLTDARC

SEQ ID NO: 45 - protein, 19 aa, Artificial sequence
  Synthetic peptide
MDMRVPAQLLGLLLLWLPG

(19) United States
(12) Patent Application Publication
(10) Pub. No.: US 2021/0393753 A1
(43) Pub. Date: Dec. 23, 2021

(54) FGL2 NEUTRALING CELL THERAPY AND METHODS OF USE THEREOF

(71) Applicant: Board of Regents, The University of Texas System, Austin, TX (US)

(72) Inventors: Shulin LI, Houston, TX (US); Jiemiao HU, Houston, TX (US); Xueqing XIA, Houston, TX (US)

(73) Assignee: Board of Regents, The University of Texas System, Austin, TX (US)

(21) Appl. No.: 17/291,303

(22) PCT Filed: Nov. 6, 2019
(86) PCT No.: PCT/US2019/060120

§ 371 Date: May 5, 2021

Related U.S. Application Data
(60) Provisional application No. 62/756,441, filed on Nov. 6, 2018.

(51) Int. Cl. A61K 39/00; A61P 35/00; C07K 16/36

(52) U.S. Cl. CPC A61K 39/0011; A61P 35/00; A61K 2039/5156; A61K 2039/505; C07K 16/36

(57) ABSTRACT

Provided herein is an FGL2 neutralization cell therapy comprising immune cells expressing a FGL2 neutralization construct. Further provided are methods for the treatment of cancer comprising administering the FGL2 neutralization cell therapy.

This application claims the benefit of U.S. Provisional Application No. 62/756,441, filed Nov. 6, 2018, the entirety of which is incorporated herein by reference.

### INCORPORATION OF SEQUENCE LISTING

The sequence listing that is contained in the file named “UTFCP1411WO_ST25.txt”, which is 16 KB (as measured in Microsoft Windows®) and was created on Nov. 5, 2019, is filed herewith by electronic submission and is incorporated by reference herein.

### BACKGROUND

### 1. Field

The present invention relates generally to the fields of immunology and medicine. More particularly, it concerns FGL2 neutralizing cell therapy and methods of use thereof.

### 2. Description of Related Art

While monoclonal antibodies targeting immune checkpoints (e.g., PD-L1, PD-1, and CTLA-4) have shown promise in the treatment of some cancers, cancers such as glioblastoma multiforme (GBM) have multiple, redundant immune-suppressive mechanisms which reduce the efficacy of immunotherapy. It may be possible that direct administration of an antibody may not be the most effective method of treatment.

Fibrinogen-like protein 2 (FGL2) is a protein that exhibits pleiotropic effects within the body and is an important immune regulator of both innate and adaptive responses. FGL2 possesses prothrombinase activity and immune regulatory functions in viral infection, allograft rejection, and abortion (Selzner et al., 2010). Some investigators have suggested that FGL2 acts as a regulatory T cell effector molecule by suppressing T cell activities in a FoxP3-dependent manner Others have found that FGL2 suppresses dendritic cell (DC) and B cell functions by binding to FcγRIIB. Furthermore, emerging data demonstrates that FGL2 regulates adaptive immunity via Th1 and Th2 cytokines. Recent studies have also shown that FGL2 can promote hepatocellular carcinoma xenograft tumor growth and angiogenesis, suggesting a tumor-promoting function.

It has been shown that FGL2 may promote GBM cancer development by inducing multiple immune-suppression mechanisms (Yan et al., 2015). The data in the Yan et al. study showed that FGL2 can function as a promoter of GBM progression by upregulating negative immune checkpoint expression and may be a therapeutic target. Thus, therapies blocking FGL2 may have a broad impact for reversing immune suppression system and may work in tumors that may not respond to other treatments. Accordingly, there is a need for therapies targeting FGL2 for the treatment of cancer.

### SUMMARY

In one embodiment, the present disclosure provides an expression construct encoding a fibrinogen-like protein 2 (FGL2) neutralization polypeptide comprising an anti-FGL2 antibody. In particular aspects, the expression construct encodes a FGL2 neutralization antibody.

In some aspects, the construct encodes an FGL2 heavy chain and an FGL2 light chain. In certain aspects, the FGL2 heavy chain comprises a first V_{H} CDR (SEQ ID NO: 5), a second V_{H} CDR (SEQ ID NO: 6), and a third V_{H} CDR (SEQ ID NO: 7). In some aspects, the FLG2 light chain comprises a first V_{L} CDR (SEQ ID NO: 8), a second V_{L} CDR (SEQ ID NO: 9), and a third V_{L} CDR (SEQ ID NO: 10). In certain aspects, the FGL2 heavy chain comprises a first V_{H} CDR identical to SEQ ID NO: 5, a second V_{H} CDR identical to SEQ ID NO: 6, and a third V_{H} CDR identical to SEQ ID NO: 7. In some aspects, the FLG2 light chain comprises a first V_{L} CDR identical to SEQ ID NO: 8, a second V_{L} CDR identical to SEQ ID NO: 9, and a third V_{L} CDR identical to SEQ ID NO: 10. The FGL2 heavy chain may have at least 90% (e.g., 91%, 92%, 93%, 94%, 95%, 96%, 97%, 98%, 99%, or 100%) identity to the amino acid sequence of SEQ ID NO:1. The FGL2 light chain may have at least 90% (e.g., 91%, 92%, 93%, 94%, 95%, 96%, 97%, 98%, 99%, or 100%) identity to the amino acid sequence of SEQ ID NO:2. In some aspects, the FGL2 heavy chain has an amino acid sequence of SEQ ID NO:1. In certain aspects, the FGL2 light chain has an amino acid sequence of SEQ ID NO:2. In some aspects, the construct comprises a FGL2 heavy chain sequence having at least 90% (e.g., 91%, 92%, 93%, 94%, 95%, 96%, 97%, 98%, 99%, or 100%) identity to SEQ ID NO:3. In certain aspects, the construct comprises a FGL2 heavy chain sequence having at least 90% (e.g., 91%, 92%, 93%, 94%, 95%, 96%, 97%, 98%, 99%, or 100%) identity to SEQ ID NO:4.

In some aspects, the scFv comprises CDRs 1-3 of the V_{H} domain and CDRs 1-3 of the V_{L} domain of the antibody encoded by hybridoma clone F48. In some aspects, the scFv comprises (a) a first V_{H} CDR at least 80% identical to V_{H} CDR1 of F48 (SEQ ID NO: 5); (b) a second V_{H} CDR at least 80% identical to V_{H} CDR2 of F48 (SEQ ID NO: 6); (c) a third V_{H} CDR at least 80% identical to V_{H} CDR3 of F48 (SEQ ID NO: 7); (d) a first V_{L} CDR at least 80% identical to V_{L} CDR1 of F48 (SEQ ID NO: 8); (e) a second V_{L} CDR at least 80% identical to V_{L} CDR2 of F48 (SEQ ID NO: 9); and (f) a third V_{L} CDR at least 80% identical to V_{L} CDR3 of F48 (SEQ ID NO: 10).

In some aspects, the scFv comprises CDRs 1-3 of the V_{H} domain and CDRs 1-3 of the V_{L} domain of the antibody encoded by hybridoma clone F48 (SEQ ID NOs:1-4) In certain aspects, the scFv comprises a V_{H} domain at least about 80% (e.g., 85%, 86%, 87%, 88%, 89%, 90%, 91%, 92%, 93%, 94%, 95%, 96%, 97%, 98%, 99%, or 100%) identical to the V_{H} domain of F48 (SEQ ID NOs:1 or 3) and a V_{L} domain at least about 80% (e.g., 85%, 86%, 87%, 88%, 89%, 90%, 91%, 92%, 93%, 94%, 95%, 96%, 97%, 98%, 99%, or 100%) identical to the V_{L} domain of F48 (SEQ ID NOs:2 or 4). In some aspects, the antibody comprises a V_{H} domain identical to the V_{H} domain of F48 (SEQ ID NOs:1 or 3) and a V_{L} domain identical to the V_{L} domain of F48 (SEQ ID NOs:2 or 4.

In certain aspects, the FLG2 heavy chain and FGL2 light chain are linked by a peptide linker, such as a “GGGGS” linker or P2A linker. For example, the GGGGS linker has an amino acid sequence of SEQ ID NO:12. In particular aspects, the construct comprises a GGGGS linker sequence of SEQ ID NO:17. In specific aspects, the P2A linker has an amino acid sequence of SEQ ID NO:14. In some aspects, the construct comprises a P2A linker sequence of SEQ ID NO:19.

In additional aspects, the construct further encodes a signal peptide. In some aspects, the signal peptide has at least 90% (e.g., 91%, 92%, 93%, 94%, 95%, 96%, 97%, 98%, 99%, or 100%) sequence identity to the amino acid sequence of SEQ ID NO:11. In some aspects, the construct comprises a signal peptide sequence having at least 90% (e.g., 91%, 92%, 93%, 94%, 95%, 96%, 97%, 98%, 99%, or 100%) identity to SEQ ID NO:16.

In further aspects, the construct further encodes a transmembrane domain, such as an EGFR transmembrane domain. The EGFR transmembrane domain may have at least 90% (e.g., 91%, 92%, 93%, 94%, 95%, 96%, 97%, 98%, 99%, or 100%) identity to amino acid sequence SEQ ID NO:15. In some aspects, the construct comprises an EGFR transmembrane domain sequence having at least 90% (e.g., 91%, 92%, 93%, 94%, 95%, 96%, 97%, 98%, 99%, or 100%) identity to SEQ ID NO:20.

In some aspects, the FGL2 neutralization antibody further comprises an Ig-Fc domain, such as an IgG-Fc fragment, such as an IgG2a-Fc. In particular aspects, the IgG2a-Fc has an amino acid sequence of at least 90% (e.g., 91%, 92%, 93%, 94%, 95%, 96%, 97%, 98%, 99%, or 100%) identity to SEQ ID NO:13. In some aspects, the construct comprises an IgG2a-Fc sequence of SEQ ID NO:18.

In particular aspects, the FGL2 neutralization antibody comprises, such as from N-terminus to C-terminus, a signal peptide, FGL2 heavy chain, peptide linker, FGL2 light chain, IgG2aFc, peptide linker, and EGFR transmembrane domain.

In some aspects, the construct is a viral vector, such as a lentiviral vector.

In another embodiment, there is provided an isolated FGL2 neutralization antibody. The FGL2 neutralization antibody may be encoded by the expression construct of the embodiments and aspects thereof. In particular aspects, the antibody is cell membrane-anchored. In some aspects, the antibody is a secreted antibody molecule.

A further embodiment provides a host cell engineered to express an FGL2 neutralization antibody, such as an antibody of the embodiments. In some aspects, the host cell is an immune cell, such as a tumor-homing cell. The cell may be a T cell, such as a peripheral blood T cell, a CD4⁺ T cell or CD8⁺ T cell. The T cell may be autologous or allogeneic. In other aspects, the immune cell is a NK cell. In particular aspects, the FGL2 neutralization antibody is anchored to the membrane of said cell.

Another embodiment provides a pharmaceutical composition comprising FGL2 neutralizing immune cells and a pharmaceutical carrier. In some aspects, the immune cells are T cells or NK cells. The FGL2 neutralizing immune cells may be engineered to express an antibody of the embodiments.

Further provided herein is a composition comprising an effective amount of FGL2 neutralizing immune cells for the treatment of cancer in a subject. In some aspects, the immune cells are T cells or NK cells. The FGL2 neutralizing immune cells may be engineered to express an antibody of the embodiments.

Also provided herein is the use of a composition comprising an effective amount of FGL2 neutralizing immune cells for the treatment of cancer in a subject. In some aspects, the immune cells are T cells or NK cells. The FGL2 neutralizing immune cells may be engineered to express an antibody of the embodiments.

A further embodiment provides the use of a composition comprising an effective amount of FGL2 neutralizing immune cells as a vaccine for the treatment of cancer in a subject. In some aspects, the immune cells are T cells or NK cells. In certain aspects, the FGL2 neutralizing immune cells are engineered to express an antibody of present embodiments. In some aspects, the vaccine induces tumor-specific resident memory T cells to prevent tumor recurrence.

In another embodiment, there is provided a method for treating cancer in a subject comprising administering an effective amount of FGL2 neutralizing immune cells to the subject. In some aspects, the immune cells are T cells or NK cells. The FGL2 neutralizing immune cells may be engineered to express an antibody of the embodiments or a fragment thereof. In particular aspects, the FGL2 neutralizing antibody is anchored to the membrane of said immune cells.

In some aspects, the FGL2 neutralizing immune cells are administered as a vaccine to induce tumor-specific resident memory T cells to prevent tumor recurrence. In specific aspects, the vaccine is administered more than once.

In some aspects, the cancer is glioblastoma, cervical cancer, pancreatic cancer, ovarian cancer, uterine cancer, esophageal cancer, melanoma cancer, head and neck cancer, colorectal cancer, bladder cancer, lung cancer, prostate cancer, sarcoma cancer, breast cancer, liver cancer, renal cancer or acute myelogenous leukemia. In particular aspects, the cancer is a FGL2-expressing cancer.

In certain aspects, the FGL2 neutralizing immune cells are administered intravenously, intracranially, intradermally, intratumorally, intramuscularly, intraperitoneally, subcutaneously, or locally.

In additional aspects, the method further comprises administering at least a second anticancer therapy to the subject. In some aspects, the second anticancer therapy is a surgical therapy, chemotherapy, radiation therapy, cryotherapy, hormonal therapy, immunotherapy or cytokine therapy. In certain aspects, second anticancer therapy is chemotherapy. In some aspects, the chemotherapy is doxorubicin or cyclophosphamide. In particular aspects, the second anticancer therapy comprises a CAR therapy, such as a CAR T cell therapy. In specific aspects, the second anticancer therapy comprises an immunomodulator, such as a STAT3 inhibitor (e.g., WP1066, S3I-201, fludarabine, TTI-101, AZD9150, COPB-31121, OPB-51602, static, niclosamine, nifuroxazide, AS1517499, C188-9, SH-4-54, napabucasin, artesunate, BP-1-1-102, cryototanshinone, SH5-07, ochromycinone, HJC0152, APTSTAT3-9R, or HO-3867), an A2AR inhibitor (e.g., SCH58261, SYN115, ZM241365, or FSPTP), or an immune checkpoint inhibitor (e.g., an anti-CTLA-4 antibody, an anti-PD-L1 antibody, and/or an anti-PD1 antibody). In some aspects, the anti-PD1 antibody is nivolumab, pembrolizumab, CT-011, BMS 936559, MPDL328OA or AMP-224. In some aspects, the second anticancer therapy comprises T cell therapy, NK cell therapy, dendritic cell therapy, or a tumor vaccine.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Schematic depicting cell membrane anchored FGL2 neutralization antibody construct. SP: signal peptide; svH: single heavy chain; svL: single light chain; GGGGS (SEQ ID NO:21) and P2xA: peptide linkers; TM: EGFR transmembrane domain. The construct can be cloned in a lentiviral for transduction to T cells or other tumor-homing cells.

FIG. 2: Schematic depicting possible mechanism of FGL2 neutralization T cell therapy. FGL2 in the tumor can induce MDSC, Tregs, macrophages, and immune checkpoints. Administering FGL2 neutralizing T cells, in which FGL2 antibody is expressed on the T cell membrane and displayed on the T cell surface, can neutralize FGL2 and boost the expansion of endogenous tumor infiltrating lymphocytes (TIL) or exogenously administered T cell expansion and tumor cell killing.

FIGS. 3A-3B: FGL2 neutralizing T cell therapy promotes efficacy of other immune therapy, such as resistance to CAR T cell therapy. The impact of FGL2 neutralization T cell therapy on CAR T cell therapy is shown. Lymphoma was treated with 2.5×10⁶ CAR T cells following doxorubicin (Dox) treatment to serve as control. The control tumor bearing mouse was euthanized due to the large tumor volume. (A) In the treatment arm the tumor bearing mouse was treated with CAR-T plus FGL2 neutralization T cells (FGL2Nu-T) (2.5 million cells each, the tumor volume was reduced initially and then stabilized. (B) In another treatment arm, the tumor bearing mouse was treated the same as control mouse (Dox+CAR-T cells), but when tumor evaded the treatment and progressed rapidly, FGL2Nu-T was administered and tumor volume declined rapidly before being stabilized.

FIG. 4: SCID mice inoculated with osteosarcoma cells to generate a patient-derived xenograft model were injected with 2.5 million FGL2 neutralizing scFv virus armed T cells [αFgl2T(892 or 921)] at days 89 and 102 post inoculation intravenously. Cyclophosphamide (Cy) was administered a few days ahead of T cell administration to assimilate the clinical application of T cell therapy. The T cells were expanded from human PBMCs. Cyclophosphamide was administered on day 81 post tumor cell inoculation at 60 mg/kg. The three treatment groups are the following: notx: no treatment; αFGL2T(892)+Cy: FGL2 neutralizing scFv virus-armed T cell treatment; CtrlT+Cy: control virus armed T cell treatment.

FIG. 5: SCID mice inoculated with osteosarcoma cells to generate a patient-derived xenograft model were injected with 2.5 million FGL2 neutralizing scFv virus armed T cells at days 85 and 92 post inoculation intravenously. The T cells were expanded from human PBMCs. Cyclophosphamide (Cy) was administered on day 79 post tumor cell inoculation at 60 mg/kg. notx: no treatment; αFGL2T(921)+Cy: FGL2 neutralizing scFv virus-armed T cell treatment; CtrlT+Cy: control virus armed T cell treatment.

FIG. 6: SCID mice inoculated with osteosarcoma cells to generate a patient-derived xenograft model were injected with 2.5 million FGL2 neutralizing scFv virus armed T cells at days 59, 71, and 83 post inoculation intravenously. The T cells were expanded from human PBMCs. Doxorubicin (Dox) was administered on days 56, 67, and 80 post tumor cell inoculation at 1 mg/kg. notx: no treatment; αFGL2T(892)+Cy: FGL2 neutralizing scFv virus-armed T cell treatment; CtrlT+Cy: control virus armed T cell treatment.

FIG. 7: NSG mice were inoculated with A549 lung tumor cells (7.5 million per mouse) subcutaneously. T cells were expanded from human PBMCs and 2.5 million T cells were armed with the FGL2 neutralizing scFv to generate the FGL2 neutralizing T cell therapy. The mice were injected intravenously on day 25 with the FGL2 neutralizing T cells. Cyclophosphamide (Cy) was administered on day 22 i.p. at a dose of 60 mg/kg. notx: no treatment; αFGL2T(921)+Cy: FGL2 neutralizing scFv virus-armed T cell treatment; CtrlT+Cy: control virus armed T cell treatment.

FIG. 8: Induction of tumor-specific memory T cells in brains using FGL2-neutralization T cell therapy. FGL2-neutralization T cell therapy eradicates DBT brain tumors, resulting in long term tumor free survivors. Intracranial rechallenge with the same tumor cells were rejected as measured by florescence on day 6.

### DESCRIPTION OF ILLUSTRATIVE EMBODIMENTS

FGL2 can induce immune checkpoint gene expression and induce regulatory T cell and macrophage accumulation in tumors. Accordingly, in certain embodiments, the present disclosure provides a FGL2 neutralization construct and a FGL2 neutralization T cell therapy. The present FGL2 neutralization cell therapy can be more effective than direct antibody administration for treating diseases such as cancer. In particular aspects, the present methods and compositions are not merely an alternative delivery method for an FGL2 antibody, but rather provide methods for direct administration of the antibody to increase efficacy for treating cancer. The method can be used to enhance immune cell therapy, such as CAR-T cell therapy. In specific aspects, the present FGL2 neutralization cell therapy has enhanced efficacy as compared to FGL2 monoclonal antibody administration.

The FGL2 neutralization T cell therapy can be used to simultaneously neutralize FGL2 and boost T cell and other immune cell activity. The FGL2 neutralization antibody may be encoded by a fusion gene fusing comprising a single chain FGL2 antibody variable encoding region (FGL2 scFv), an Ig-Fc encoding region, and a cell transmembrane encoding domain. The gene may be cloned into a viral vector. The virus expressing this fusion gene may be transfected into immune cells, such as T cells, or apoptotic tumor cells for homing to tumors. In specific aspects, the FGL2 neutralization antibody is membrane-anchored to the host cell, such as T cells, such as through the EGFR transmembrane domain. The FGL2 neutralization cell therapy provided herein can enhance anti-tumor activity alone or boost other cell therapy, such as CAR T cell therapy, NK cell therapy, or dendritic cell therapy. Other combination therapies may comprise macrophages, tumor cells, or tumor vaccines. The present methods may be used for treating multiple cancers, including glioblastoma, lung cancer, and melanoma, alone or in combination with other anti-cancer therapies. The anti-cancer therapies may include immune checkpoint inhibitors, such as an anti-PDL1 antibody, and/or chemotherapy, such as TMZ, cyclophosphamide, or doxorubicin.

In further aspects, the present FGL2 neutralization therapy, such as FGL2 neutralization T cell therapy, may be used as a tumor vaccine to induce tumor-specific resident memory T cells to prevent tumor relapse.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

### II. FGL2 NEUTRALIZING CONSTRUCT

Certain embodiments of the present disclosure concern FGL2 neutralizing antibodies. A “neutralizing antibody” or “neutralization antibody” as used herein refers to an antibody that neutralizes the biological activity of Fgl2 that suppresses immune surveillance against tumor cells.

A. FGL2 Heavy and Light Chains

The FGL1 neutralizing antibody may comprise an antibody or a fragment thereof that binds to at least a portion of FGL2 protein and inhibits FGL2 signaling. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. Preferably, the FGL2 antibody is a monoclonal antibody or a humanized antibody.

In some embodiments, the FGL2 scFv comprises CDRs 1-3 of the heavy chain of SEQ ID NO:1 (SYWMQ; EIDPSDSYTNYNQKFKG; NGNYYGSTYDY (SEQ ID NOs:5-7)) and the CDRs 1-3 of the light chain of SEQ ID NO:2 (RASQDVSNYLN; YTSRLHS; QQGNTLPPWT (SEQ ID NOs:8-10)). The anti-FGL2 scFv may have at least 80%, such as 85%, 90%, 91%, 92%, 93%, 94%, 95%, 96%, 97%, 98%, 99%, or 100%, sequence identity to SEQ ID NOs:1-4.

FGL2 mAB Heavy chain:
(SEQ ID NO: 1)
QVQLQQPGAELVKPGASVKLSCKASGYTFASYWMQWVKQRPGQGL
EWIGEIDPSDSYTNYNQKFKGKATLTVDTSSNTAYMQLSSLTSED
SAVYYCARNGNYYGSTYDYWGQGTTLTVSS
FGL2 mAB Light chain:
(SEQ ID NO: 2)
DIQMTQTTSSLSASLGDRVTISCRASQDVSNYLNWYQQKPDGSVK
LLIYYTSRLHSGVPSRFSGSGSGAHYSLTISNLEQEDIATYFCQQ
GNTLPPWTFGGGTKLEIK
FGL2 mAB Heavy chain:
(SEQ ID NO: 3)
CAGGTCCAACTGCAGCAGCCTGGGGCTGAGCTTGTGAAGCCTGGG
GCTTCAGTGAAGCTGTCCTGCAAGGCTTCTGGCTACACCTTCGCC
AGCTACTGGATGCAGTGGGTAAAACAGAGGCCTGGACAGGGCCTT
GAGTGGATCGGAGAGATTGATCCTTCTGATAGCTATACTAACTAC
AATCAAAAGTTCAAGGGCAAGGCCACATTGACTGTAGACACATCC
TCCAACACAGCCTACATGCAGCTCAGCAGCCTGACATCTGAGGAC
TCTGCGGTCTATTACTGTGCAAGAAATGGGAATTACTACGGTAGT
ACCTACGACTACTGGGGCCAAGGCACCACTCTCACAGTCTCCTCA
FGL2 mAB Light chain:
(SEQ ID NO: 4)
GATATCCAGATGACACAGACTACATCCTCCCTGTCTGCCTCTCTG
GGAGACAGAGTCACCATCAGTTGCAGGGCAAGTCAGGACGTTAGC
AATTATTTAAACTGGTATCAGCAGAAACCAGATGGATCTGTTAAA
CTCCTGATCTACTACACTTCAAGATTACACTCAGGAGTCCCATCA
AGGTTCAGTGGCAGTGGGTCTGGAGCACATTATTCTCTCACCATT
AGCAACCTGGAGCAAGAAGATATTGCCACTTACTTTTGCCAACAG
GGTAATACGCTTCCTCCGTGGACGTTCGGTGGAGGCACCAAGCTG
GAAATCAAG

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that a bacteria containing such a variant may be implemented in compositions and methods. Consequently, a protein need not be isolated.

It is contemplated that in compositions there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds FGL2.

An antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Embodiments provide antibodies and antibody-like molecules against FGL2, polypeptides and peptides that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

B. Peptide Linkers

Peptide linkers known in the art for fusion proteins may be used in the present FGL2 neutralizing antibody, such as to fuse the heavy and light chain or fuse the transmembrane domain. These linker peptides serve to connect the protein moieties, and also provide many other functions, such as maintaining cooperative inter-domain interactions or preserving biological activity.

Flexible linkers may be usually used when the joined domains require a certain degree of movement or interaction. They can be generally composed of small, non-polar (e.g. Gly) or polar (e.g. Ser or Thr) amino acids. The small size of these amino acids provides flexibility, and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties.

Exemplary linkers include, but are not limited to:

GGGGS Linker:
(SEQ ID NO: 12)
GGGGSGGGGSGGGGS
GGGS Linker:
(SEQ ID NO: 17)
GGGGGCGGCGGATCCGGGGGAGG
GGGTTCTGGCGGAGGTGGGTCC
P2A linker
(SEQ ID NO: 14)
(GSG)ATNFSLLKQAGDVEENPGP
P2A linker
(SEQ ID NO: 19)
GGGAGCGGAGCGACGAATTTCAGC
CTGCTGAAACAGGCTGGAGATGTG
GAGGAGAACCCGGGC

The most commonly used flexible linkers have sequences consisting primarily of stretches of Gly and Ser residues (“GS” linker). An example of a flexible linker has the sequence of (Gly-Gly-Gly-Gly-Ser)ₙ (SEQ ID NO:21), such as SEQ ID NO:12. By adjusting the copy number “n”, the length of this GS linker can be optimized to achieve appropriate separation of the functional domains, or to maintain necessary inter-domain interactions. Besides the GS linkers, many other flexible linkers have been designed for recombinant fusion proteins. These flexible linkers may be rich in small or polar amino acids such as Gly and Ser, but can contain additional amino acids such as Thr and Ala to maintain flexibility, as well as polar amino acids such as Lys and Glu to improve solubility. Other types of flexible linkers, including KESGSVSSEQLAQFRSLD (SEQ ID NO:38) and EGKSSGSGSESKST (SEQ ID NO:39), (Gly)₈ (SEQ ID NO:22), or GSAGSAAGSGEF (SEQ ID NO:40), may be applied for the construction of the scFv.

While flexible linkers have the advantage to connect the functional domains passively and permitting certain degree of movements, rigid linkers may also be used. Alpha helix-forming linkers with the sequence of (EAAAK)n (SEQ ID NO:25) or a Pro-rich sequence, (XP)n, with X designating any amino acid, preferably Ala, Lys, or Glu, may be used.

Cleavable linkers may be used for the present FGL2 neutralization antibody. This type of linker may reduce steric hindrance, improve bioactivity, or achieve independent actions/metabolism of individual domains of recombinant fusion proteins after linker cleavage.

TABLE 1
Exemplary peptide linkers.

| | Examples | | | |
|---|---|---|---|---|
| | | Linker | | |
| | | | | SEQ |
| Linker | Fusion | | | ID |
| Function | Protein | Type | Sequence^{a} | NO |
| | scFv | flexible | (GGGGS)₃ | 12 |
| | G-CSF-Tf | flexible | (GGGGS)₃ | 12 |
| | HBsAg | flexible | (GGGGS)₃ | 12 |
| | preS1 | | | |
| Increase | Myc- | flexible | (Gly)₈ | 22 |
| Stability/ | Est2p | | | |
| Folding | | | | |
| | albumin-ANF | flexible | (Gly)₆ | 23 |
| | virus coat | rigid | (EAAAK)₃ | 24 |
| | protein | | | |
| | beta- | rigid | (EAAAK)n | 25 |
| | glucanase- | | (n = 1-3) | |
| | xylanase | | | |
| Increase | hGH-Tf | rigid | A(EAAAK)₄ALE | 26 |
| expression | and Tf-hGH | | A(EAAAK)₄A | |
| | G-CSF-Tf | | | |
| | and | | | |
| | Tf-CSF-Tf | | A(EAAAK)₄ALE | 26 |
| | | | A(EAAAK)₄A | |
| | G-CSF-Tf | flexible | (GGGGS)₃ | 12 |
| | G-CSF-Tf | rigid | A(EAAAK)₄ALE | 26 |
| | | | A(EAAAK)₄A | |
| | hGH-Tf | rigid | A(EAAAK)₄ALE | 26 |
| | | | A(EAAAK)₄A | |
| Improve | HSA-IFN- | flexible | GGGGS | 21 |
| biological | α2b | | | |
| activity | | | | |
| | HSA-IFN- | rigid | PAPAP | 27 |
| | α2b | | | |
| | HSA-IFN- | rigid | AEAAAKEAAA | 28 |
| | α2b | | KA | |
| | PGA-rTHS | flexible | (GGGGS)ₙ | 21 |
| | | | (n = 1, | |
| | | | 2, 4) | |
| | interferon- | rigid | (Ala-Pro)ₙ | |
| | γ-gp120 | | (10-34 aa) | |
| | GSF-S-S-Tf | cleavable | disulfide | |
| | IFN-α2b-HSA | cleavable | disulfide | |
| | FIX-albumin | cleavable | VSQTSKLTRA | 29 |
| | | | ETVFPDV^{b} | |
| | LAP-IFN-β | cleavable | PLG ↓ LWA^{c} | 30 |
| Enable | MazE-MazF | cleavable | RVL↓AEA; | 31; |
| targeting | | | EDVVCC↓S | 32 |
| | | | MSY; | 33 |
| | | | GGIEGR↓G↓S^{c} | |
| | | | TRHRQPR↓ | 34 |
| | | | GWE; | |
| | Immuno- | cleavable | AGNRVRR↓SVG; | 35 |
| | toxins | | RRRRRRR↓R↓R^{d} | |
| | Immuno- | cleavable | GFLG↓^{e} | 37 |
| | toxin | dipeptide | LE | |
| Alter Pk | G-CSF-Tf | rigid | A(EAAAK)₄ALE | 26 |
| | and | | A(EAAAK)₄A | |
| | hGH-Tf | cleavable | Disulfide | |

C. Signal Peptide

The FGL2 neutralization antibody may comprise one or more signal peptides for secretion. The signal peptide may be 16-30 amino acids and can be at the N-terminus of the neutralizing antibody. The signal peptide may comprise a positively charged N-terminus, referred to as a basic amino terminus; an intermediate hydrophobic sequence, neutral amino acid-based, a spiral structure section can be formed, which is the main function of the signal peptide region; a C-terminus with negative charge, small molecules containing amino acids, signal sequence cleavage site, also known as processing zones. An exemplary signal peptide comprises:

Signal peptide
(SEQ ID NO: 16)
CCACCATGGGATGGAGCTGTATCATCCTCTTCTTGGTAGCAAC
AGCTACAGGTGTCCACTCT
Signal peptide
(SEQ ID NO: 11)
MGWSCIILFLVATATGVHS

Other exemplary signal peptides that may be used include, but are not limited to, MGKWVKVLFALICIAVAES (SEQ ID NO:41), METPAQLLFLLLLWLP (SEQ ID NO:42), MGWSCIILFLVATATG (SEQ ID NO:43), MSVPTQVLGLLLLWLTDARC (SEQ ID NO:44), and MDMRVPAQLLGLLLLWLPG (SEQ ID NO:45).

D. Transmembrane Domain

The FGL2 neutralizing antibody may comprise a transmembrane domain, such as to anchor the antibody to a cell. Any transmembrane domain known in the art may be used for the membrane-anchored expression of the FGL2 neutralizing construct to the host cell, such as T cells. An exemplary transmembrane domain is the EGFR transmembrane domain

Transmembrane domain:
(SEQ ID NO: 15)
SIATGMVGALLLLLVVALGIGLFMRRRHIVRKRTLRRLLQEREL
Transmembrane domain:
(SEQ ID NO: 20)
TCCATCGCC ACTGGGATGG TGGGGGCCCT CCTCTTGCTG
CTGGTGGTGG CCCTGGGGAT CGGCCTCTTCATG CGAAGGCGCC
ACATCGTTCG GAAGCGCACG CTGCGGAGGC TGCTGCAGGA
GAGGGAGCTTTGA

E. Fc Domain

The FGL2 neutralizing construct may comprise an Ig-Fc domain, such as a human IgA-Fc domain, IgM-Fc domain, or IgG-Fc domain, such as IgG1, IgG2, IgG3, or IgG4, or a fragment thereof. In specific aspects, the Fc domain is IgG2a-Fc domain, such as SEQ ID NO:13. In other aspects, the FGL2 neutralizing construct does not comprise an Ig-Fc domain

IgG2aFC
(SEQ ID NO: 13)
PRGPTIKPCPPCKCPAPNLLGGPSVFIFPPKIKDVLMISLSPI
VTCVVVDVSEDDPDVQISWFVNNVEVHTAQTQTHREDYNSTLR
VVSALPIQHQDWMSGKEFKCKVNNKDLPAPIERTISKPKGSVR
APQVYVLPPPEEEMTKKQVTLTCMVTDFMPEDIYVEWTNNGKT
ELNYKNTEPVLDSDGSYFMYSKLRVEKKNWVERNSYSCSVVHE
GLHNHHTTKSFSRTPGK
IgG2aFC
(SEQ ID NO: 18)
CCCAGAGGGCCCACAATCAAGCCCTGTCCTCCATGCAAATGCC
CAGCACCTAACCTCTTGGGTGGACCATCCGTCTTCATCTTCCC
TCCAAAGATCAAGGATGTACTCATGATCTCCCTGAGCCCCATA
GTCACATGTGTGGTGGTGGATGTGAGCGAGGATGACCCAGATG
TCCAGATCAGCTGGTTTGTGAACAACGTGGAAGTACACACAGC
TCAGACACAAACCCATAGAGAGGATTACAACAGTACTCTCCGG
GTGGTCAGTGCCCTCCCCATCCAGCACCAGGACTGGATGAGTG
GCAAGGAGTTCAAATGCAAGGTCAACAACAAAGACCTCCCAGC
GCCCATCGAGAGAACCATCTCAAAACCCAAAGGGTCAGTAAGA
GCTCCACAGGTATATGTCTTGCCTCCACCAGAAGAAGAGATGA
CTAAGAAACAGGTCACTCTGACCTGCATGGTCACAGACTTCAT
GCCTGAAGACATTTACGTGGAGTGGACCAACAACGGGAAAACA
GAGCTAAACTACAAGAACACTGAACCAGTCCTGGACTCTGATG
GTTCTTACTTCATGTACAGCAAGCTGAGAGTGGAAAAGAAGAA
CTGGGTGGAAAGAAATAGCTACTCCTGTTCAGTGGTCCACGAG
GGTCTGCACAATCACCACACGACTAAGAGCTTCTCCCGGACTC
CGGGTAAA

F. T Cell Therapy

Certain embodiments of the present disclosure concern obtaining and administering T cells to a subject as an immunotherapy to target cancer cells, such as T cells engineered to express the FGL2 neutralizing antibody provided herein. Several basic approaches for the derivation, activation and expansion of functional anti-tumor effector T cells have been described in the last two decades. These include: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization which can be used in the methods of the present disclosure.

In some embodiments, the T cells are derived from the blood, cord blood, bone marrow, lymph, or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells). In one embodiment, the cells (e.g., CD8⁺ cells or CD3⁺ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In some examples, CD8⁺ T cells are enriched for cells positive for CD45RO (or negative for CD45RA) and for CD62L.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days. For example, the cells may be cultured from 5 days, 5.5 days, or 5.8 days to 21 days, 21.5 days, or 21.8 days, such as from 10 days, 10.5 days, or 10.8 days to 14 days, 14.5 days, or 14.8 days.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days, preferably about 14 days. More preferably, rapid expansion provides an increase of at least about 200-fold (e.g., 200-, 300-, 400-, 500-, 600-, 700-, 800-, 900-, or greater) over a period of about 10 to about 14 days, preferably about 14 days.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being preferred. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

The autologous T-cells can be modified to express a T-cell growth factor that promotes the growth and activation of the autologous T-cells. Suitable T-cell growth factors include, for example, interleukin (IL)-2, IL-7, IL-15, and IL-12. Suitable methods of modification are known in the art. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, N Y, 1994. In particular aspects, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression.

G. Methods of Delivery

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

a. Viral Vectors

Viral vectors encoding an antigen receptor may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

b. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

(i) Promoter/Enhancers

The expression constructs provided herein comprise a promoter to drive expression of the antigen receptor. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30110 bp—upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the βlactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

(ii) Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth diease virus 2A) or a “2A-like” sequence (e.g., Thosea asigna virus 2A; T2A).

(iii) Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

c. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

d. Other Methods of Nucleic Acid Delivery

In addition to viral delivery of the nucleic acids encoding the antigen receptor, the following are additional methods of recombinant gene delivery to a given host cell and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

### III. METHODS OF TREATMENT

Certain aspects of the present embodiments can be used to prevent or treat a disease or disorder associated with FGL2 signaling. Signaling of FGL2 may be reduced by any suitable drugs to prevent cancer cell proliferation. Preferably, such substances would be FGL2 neutralization construct to reverse mechanisms which suppress the immune system.

In certain embodiments, the compositions and methods of the present embodiments involve a neutralizing antibody against FGL2 to inhibit its activity in cancer cell proliferation, which may be administered in combination with a second or additional therapy. Such therapy can be applied in the treatment of any disease that is associated with FGL2-mediated cell proliferation. For example, the disease may be cancer.

In some embodiments, the present disclosure provides methods for immunotherapy comprising administering an effective amount of the FGL2-neutralizing T cells of the present disclosure. In one embodiment, a medical disease or disorder is treated by transfer of a FGL2-neutralizing T cell population that elicits an immune response. In certain embodiments of the present disclosure, cancer is treated by transfer of a FGL2-neutralizing T cell population that elicits an immune response. Provided herein are methods for treating or delaying progression of cancer in an individual comprising administering to the individual an effective amount FGL2-neutralizing T cell therapy. The present methods may be applied for the treatment of immune disorders, solid cancers, and hematologic cancers. Specifically, the cancer may be glioblastoma.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

In some embodiments of the methods of the present disclosure, the treated T cells in the individual are characterized by lowing the exhausting gene expression; the activated CD4 and/or CD8 T cells in the individual are characterized by γ-IFN producing CD4 and/or CD8 T cells and/or enhanced cytolytic activity relative to prior to the administration of the combination. γ-IFN may be measured by any means known in the art, including, e.g., intracellular cytokine staining (ICS) involving cell fixation, permeabilization, and staining with an antibody against γ-IFN. Cytolytic activity may be measured by any means known in the art, e.g., using a cell killing assay with mixed effector and target cells. Likewise, myeloid cells in the treated individual are characterized by lowing immune suppressive gene expression.

In some embodiments, the subject can be administered nonmyeloablative lymphodepleting chemotherapy prior to the FGL2-neutralizing T cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a T cell growth factor that promotes the growth and activation of the autologous T cells is administered to the subject either concomitantly with the autologous T cells or subsequently to the autologous T cells. The T cell growth factor can be any suitable growth factor that promotes the growth and activation of the autologous T cells. Examples of suitable T-cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2. IL-12 is a preferred T-cell growth factor.

Therapeutically effective amounts of immune cells can be administered by a number of routes, including parenteral administration, for example, intravenous, intraperitoneal, intramuscular, intracranial, intrasternal, or intraarticular injection, or infusion.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

The T cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of T cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ T cells/m². In a certain embodiment, the dose used in the treatment of human subjects ranges from about 3.8×10⁹ to about 3.8×10¹⁰ T cells/m². In additional embodiments, a therapeutically effective amount of T cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of T cells is readily determined by one of skill in the art based on the age, weight, sex, and physiological condition of the subject. Effective doses can be extrapolated from dose-response curves derived from in vitro or animal model test systems.

A. Pharmaceutical Compositions

Also provided herein are pharmaceutical compositions and formulations comprising FGL2-neutralizing T cells and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn— protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

B. Combination Therapies

In certain embodiments, the compositions and methods of the present embodiments involve a FGL2-neutralizing T cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below FGL2-neutralizing T cell therapy is “A” and an anti-cancer therapy is “B”:

A/B/A B/A/B B/B/A A/A/B A/B/B
B/A/A A/B/B/B B/A/B/B B/B/B/A
B/B/A/B A/A/B/B A/B/A/B A/B/B/A
B/B/A/A B/A/B/A B/A/A/B A/A/A/B
B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates (ADCs) comprise monoclonal antibodies (MAbs) that are covalently linked to cell-killing drugs and may be used in combination therapies. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. Exemplary ADC drugs inlcude ADCETRIS® (brentuximab vedotin) and KADCYLA® (trastuzumab emtansine or T-DM1).

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, erb b2 and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds); cytokine therapy, e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (AZAR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies. Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody that may be used. Pembrolizumab, also known as MK-3475, Merck 3475, lambrolizumab, KEYTRUDA®, and SCH-900475, is an exemplary anti-PD-1 antibody. CT-011, also known as hBAT or hBAT-1, is also an anti-PD-1 antibody. AMP-224, also known as B7-DCIg, is a PDL2-Fc fusion soluble receptor.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. An exemplary anti-CTLA-4 antibody is ipilimumab (also known as 10D1, MDX-010, MDX-101, and Yervoy®) or antigen binding fragments and variants thereof. In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin.

### IV. ARTICLES OF MANUFACTURE OR KITS

An article of manufacture or a kit is provided comprising FGL2 neutralizing antibody or FGL2-neutralizing T cells is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—FGL2 Neutralization Antibody Development and Characterization

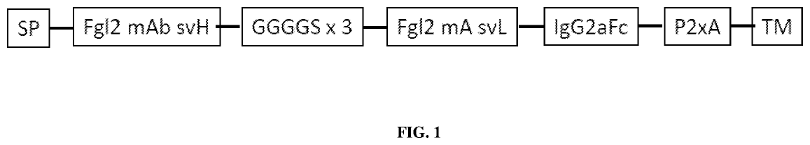

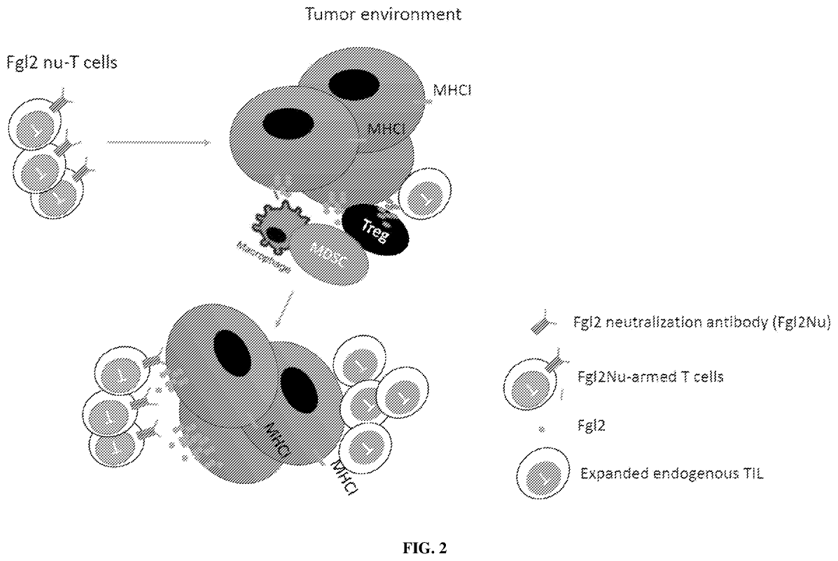

Cell membrane anchored FGL2 neutralization antibody was developed by linking a signal peptide, single heavy chain, single light chain, peptide linkers, and an EGFR transmembrane domain as depicted in FIG. 1. The construct was then cloned into a lentiviral for transduction to T cells or other tumor-homing cells.

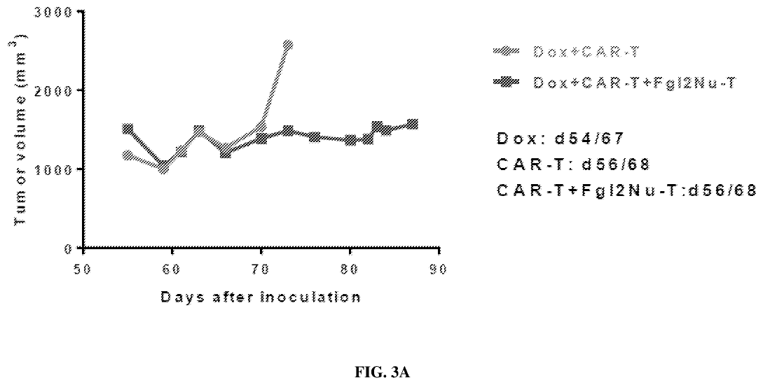

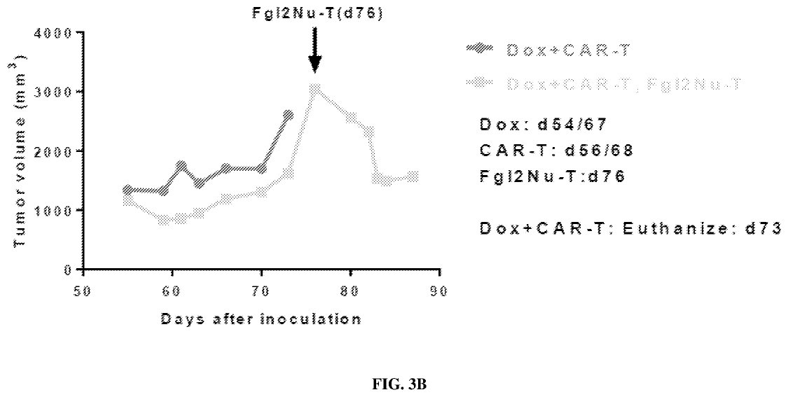

The impact of FGL2 neutralization T cell therapy on CAR T cell therapy was determined. A PDX sarcoma was treated with 2.5×10⁶ CAR T cells following doxorubicin (Dox) treatment to serve as control. The control tumor bearing mouse was euthanized due to the large tumor volume. In the treatment arm, the tumor bearing mouse was treated with CAR-T plus FGL2 neutralization T cells (FGL2Nu-T) (2.5 million cells each). It was observed that the tumor volume was reduced initially and then stabilized (FIG. 3A). In another treatment arm, the tumor bearing mouse was treated the same as control mouse (Dox+CAR-T cells), but when the tumor evaded the treatment and progressed rapidly, FGL2Nu-T was administered and tumor volume declined rapidly before being stabilized (FIG. 3B).

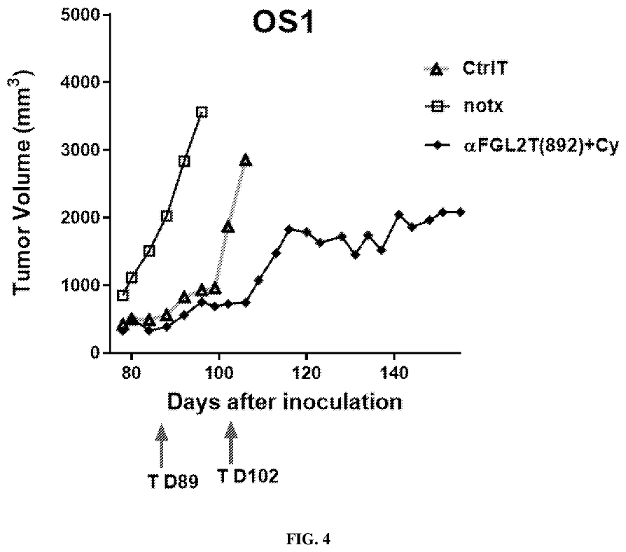

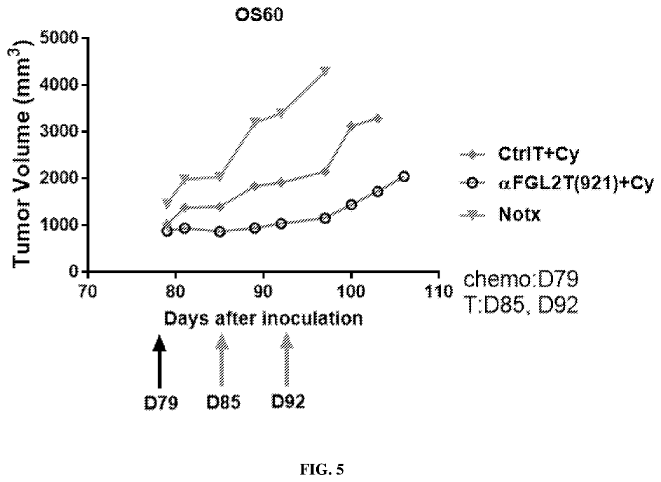

Further mice studies were performed to characterize the FGL2 neutralization T cell therapy. SCID mice inoculated with osteosarcoma cells to generate a patient-derived xenograft model were injected with 2.5 million FGL2 neutralizing scFv virus armed T cells at days 89 and 102 post inoculation intravenously (FIG. 4). The T cells were expanded from human PBMCs. Cyclophosphamide was administered on day 81 post tumor cell inoculation at 60 mg/kg. Next, the PDX sarcoma model was injected with 2.5 million FGL2 neutralizing scFv virus armed T cells at days 85 and 92 post inoculation intravenously (FIG. 5). The T cells were expanded from human PBMCs. Cyclophosphamide was administered on day 79 post tumor cell inoculation at 60 mg/kg. It was observed that the FGL2 neutralizing T cells in combination with the cyclophosphamide resulted in reduced tumor volume.

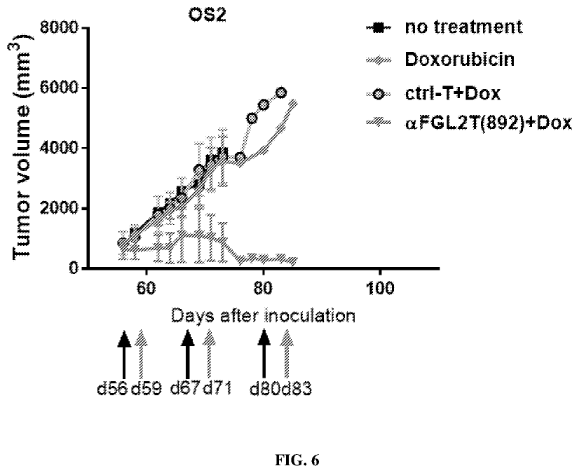

In addition, SCID mice inoculated with osteosarcoma cells to generate a patient-derived xenograft model were injected with 2.5 million FGL2 neutralizing scFv virus armed T cells at days 59, 71, and 83 post inoculation intravenously (FIG. 6). The T cells were expanded from human PBMCs. Doxorubicin was administered on days 56, 67, and 80 post tumor cell inoculation at 1 mg/kg. The combination of the FGL2 neutralizing T cells in combination with doxorubicin resulted in almost complete reduction in tumor volume.

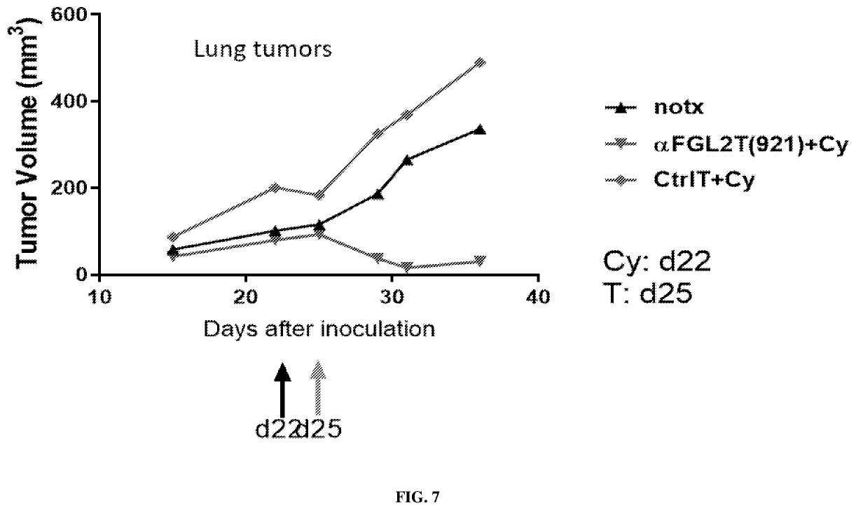

Next, NSG mice were inoculated with A549 lung tumor cells (7.5 million per mouse) subcutaneously. T cells were expanded from human PBMCs and 2.5 million T cells were armed with the FGL2 neutralizing scFv to generate the FGL2 neutralizing T cell therapy (FIG. 7). The mice were injected intravenously on day 25 with the FGL2 neutralizing T cells. Cyclophosphamide was administered on day 22 i.p. at a dose of 60 mg/kg. The combination of the FGL2 neutralizing T cells and cyclophosphamide at an earlier tumor point resulted in almost complete inhibition of tumor growth.

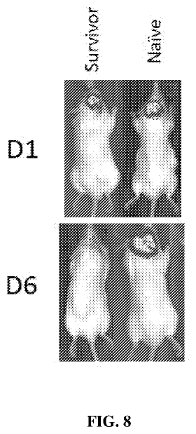

Finally, induction of tumor-specific memory T cells in brains was observed using FGL2-neutralization T cell therapy. FGL2-neutralization T cell therapy eradicates DBT brain tumors, resulting in survivors. Intracranial rechallenge with the same tumor cells were rejected as measured by florescence on day 6 (FIG. 8). Thus, this cell therapy may also act as a vaccine to induce tumor-specific memory T cells in brains.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Ausubel et al., Current Protocols in Molecular Biology, Greene Publishing Associates and John Wiley & Sons, N Y, 1994.
Kabat et al., Sequences of Proteins of Immunological Interest, National Institutes of Health, Bethesda, Md., 1987.
Sambrook et al., Molecular Cloning: A Laboratory Manual, 3^{rd} ed., Cold Spring Harbor Press, Cold Spring Harbor, N.Y. 2001.
Selzner et al., Rambam Maimonides Med J., 1(1):e0004, 2010.
Terakura et al. Blood. 1:72-82, 2012.
U.S. Pat. No. 5,994,136
U.S. Pat. No. 6,013,516
Wang et al. J Immunother. 35(9):689-701, 2012.
Yan et al., J Natl Cancer Inst, 107(8), 2015.

1. An expression construct encoding a fibrinogen-like protein 2 (FGL2) neutralization polypeptide comprising anti-FGL2 antibody.

2. The construct of claim 1, wherein the antibody is selected from the group consisting of F(ab′)2, Fab′, Fab, Fv, and scFv.

3. The construct of claim 1, wherein the antibody is a scFv.

4. The construct of any of claims 1-3, wherein the construct encodes an FGL2 heavy chain and an FGL2 light chain.

5. The construct of claim 4, wherein the FGL2 heavy chain comprises a first V_{H} CDR (SEQ ID NO: 5), a second V_{H} CDR (SEQ ID NO: 6), and a third V_{H} CDR (SEQ ID NO: 7).

6. The construct of claim 4 or 5, wherein the FLG2 light chain comprises a first V_{L} CDR (SEQ ID NO: 8), a second V_{L} CDR (SEQ ID NO: 9), and a third V_{L} CDR (SEQ ID NO: 10).

7. The construct of any of claims 1-6, wherein the FGL2 heavy chain has at least 90% identity to the amino acid sequence of SEQ ID NO:1.

8. The construct of any of claims 1-7, wherein the FGL2 light chain has at least 90% identity to the amino acid sequence of SEQ ID NO:2.

9. The construct of any of claims 1-8, wherein the FGL2 heavy chain has an amino acid sequence of SEQ ID NO:1.

10. The construct of any of claims 1-9, wherein the FGL2 light chain has an amino acid sequence of SEQ ID NO:2.

11. The construct of any of claims 1-10, wherein the construct comprises a FGL2 heavy chain sequence having at least 90% identity to SEQ ID NO:3.

12. The construct of any of claims 1-11, wherein the construct comprises a FGL2 heavy chain sequence having at least 90% identity to SEQ ID NO:4.

13. The construct of any of claims 1-12, wherein the FLG2 heavy chain and FGL2 light chain are linked by a peptide linker.

14. The construct of claim 13, wherein the peptide linker is a GGGGS linker or P2A linker.

15. The construct of claim 14, wherein the GGGGS linker has an amino acid sequence of SEQ ID NO:12.

16. The construct of claim 14 or 15, wherein the construct comprises a GGGGS linker sequence of SEQ ID NO:17.

17. The construct of any of claims 14-16, wherein the P2A linker has an amino acid sequence of SEQ ID NO:14.

18. The construct of any of claims 14-17, wherein the construct comprises a P2A linker sequence of SEQ ID NO:19.

19. The construct of any of claims 1-18, wherein the construct further encodes a signal peptide.

20. The construct of claim 19, wherein the signal peptide has at least 90% sequence identity to the amino acid sequence of SEQ ID NO:11.

21. The construct of claim 19 or 20, wherein the signal peptide has an amino acid sequence of SEQ ID NO:11.

22. The construct of any of claims 1-21, wherein the construct comprises a signal peptide sequence having at least 90% identity to SEQ ID NO:16.

23. The construct of any of claims 1-22, wherein the construct comprises a signal peptide sequence of SEQ ID NO:16.

24. The construct of any of claims 1-23, wherein construct further encodes a transmembrane domain.

25. The construct of claim 24, wherein the transmembrane domain is an EGFR transmembrane domain.

26. The construct of claim 25, wherein the EGFR transmembrane domain has at least 90% identity to amino acid sequence SEQ ID NO:15.

27. The construct of claim 25 or 26, wherein the EGFR transmembrane domain has an amino acid sequence of SEQ ID NO:15.

28. The construct of any of claims 1-27, wherein the construct comprises an EGFR transmembrane domain sequence having at least 90% identity to SEQ ID NO:20.

29. The construct of any of claims 1-28, wherein the construct comprises an EGFR transmembrane domain sequence of SEQ ID NO:20.

30. The construct of any of claims 1-29, wherein the FGL2 neutralization antibody further comprises an Ig-Fc domain or fragment thereof.

31. The construct of claim 30, wherein the Ig-Fc domain is an IgG-Fc fragment.

32. The construct of claim 30, wherein the Ig-Fc domain is IgG2a-Fc.

33. The construct of claim 32, wherein the IgG2a-Fc has an amino acid sequence of SEQ ID NO:13.

34. The construct of claim 32, wherein the construct comprises an IgG2a-Fc sequence of SEQ ID NO:18.

35. The construct of any of claims 1-34, wherein the FGL2 neutralization antibody comprises a signal peptide, FGL2 heavy chain, peptide linker, FGL2 light chain, IgG2aFc, peptide linker, and EGFR transmembrane domain.

36. The construct of any of claims 1-35, wherein the FGL2 neutralization antibody comprises from N-terminus to C-terminus a signal peptide, FGL2 heavy chain, peptide linker, FGL2 light chain, IgG2aFc, peptide linker, and EGFR transmembrane domain.

37. The construct of any of claims 1-36, wherein the construct is a viral vector.

38. The construct of claim 37, wherein the viral vector is a lentiviral vector.

39. An isolated FGL2 neutralization antibody, wherein the FGL2 neutralization antibody is encoded by the expression construct of any one of claims 1-38.

40. The antibody of claim 39, wherein the antibody is cell membrane-anchored.

41. The antibody of claim 39, wherein the antibody is a secreted antibody molecule.

42. A host cell engineered to express the FGL2 neutralization antibody of claim 39 or the construct of any one of claims 1-38.

43. The host cell of claim 42, wherein the host cell is an immune cell.

44. The host cell of claim 43, wherein the immune cell is a tumor-homing cell.

45. The host cell of claim 43 or 44, wherein the immune cell is a T cell.

46. The host cell of claim 45, wherein the T cell is a peripheral blood T cell.

47. The host cell of claim 45 or 46, wherein the T cell is a CD4⁺ T cell or CD8⁺ T cell.

48. The host cell of any of claims 45-47, wherein the T cell is autologous.

49. The host cell of any of claims 45-47, wherein the T cell is allogeneic.

50. The host cell of claim 42, wherein the immune cell is a NK cell.

51. The host cell of any of claims 42-50, wherein the FGL2 neutralization antibody is anchored to the membrane of said cell.

52. A pharmaceutical composition comprising FGL2 neutralizing immune cells and a pharmaceutical carrier.

53. The composition of claim 52, wherein the immune cells are T cells.

54. The composition of claim 52, wherein the immune cells are NK cells.

55. The composition of claim 52, wherein the FGL2 neutralizing immune cells are engineered to express an antibody of claim 39 or claim 40 or a construct of any of claims 1-38.

56. A composition comprising an effective amount of FGL2 neutralizing immune cells for the treatment of cancer in a subject.

57. The composition of claim 56, wherein the immune cells are T cells or NK cells.

58. The composition of claim 56 or 57, wherein the FGL2 neutralizing immune cells are engineered to express an antibody of claim 39 or fragment thereof.

59. The use of a composition comprising an effective amount of FGL2 neutralizing immune cells for the treatment of cancer in a subject.

60. The use of claim 59, wherein the immune cells are T cells or NK cells.

61. The use of claim 59 or 60, wherein the FGL2 neutralizing immune cells are engineered to express an antibody of claim 39.

62. The use of a composition comprising an effective amount of FGL2 neutralizing immune cells as a vaccine for the treatment of cancer in a subject.

63. The use of claim 62, wherein the immune cells are T cells or NK cells.

64. The use of claim 62 or 63, wherein the FGL2 neutralizing immune cells are engineered to express an antibody of claim 39.

65. The use of any of claims 62-64, wherein the vaccine induces tumor-specific resident memory T cells to prevent tumor recurrence.

66. A method for treating cancer in a subject comprising administering an effective amount of FGL2 neutralizing immune cells to the subject.

67. The method of claim 66, wherein the immune cells are T cells.

68. The method of claim 66, wherein the immune cells are NK cells.

69. The method of any of claims 66-68, wherein the FGL2 neutralizing immune cells are engineered to express an antibody of claim 39 or a construct of any one of claims 1-38.

70. The method of any of claims 66-69, wherein the FGL2 neutralizing immune cells are administered as a vaccine to induce tumor-specific resident memory T cells to prevent tumor recurrence.

71. The method of claim 70, wherein the vaccine is administered more than once.

72. The method of any of claims 66-69, wherein the FGL2 neutralizing antibody is anchored to the membrane of said immune cells.

73. The method of any of claims 66-72, wherein the cancer is glioblastoma, cervical cancer, pancreatic cancer, ovarian cancer, uterine cancer, esophageal cancer, melanoma cancer, head and neck cancer, colorectal cancer, bladder cancer, lung cancer, prostate cancer, sarcoma cancer, breast cancer, liver cancer, renal cancer or acute myelogenous leukemia.

74. The method of any of claims 66-73, wherein the cancer is glioblastoma.

75. The method of any of claims 66-74, wherein the cancer is a FGL2-expressing cancer.

76. The method of any of claims 66-75, wherein the FGL2 neutralizing immune cells are administered intravenously, intradermally, intratumorally, intramuscularly, intraperitoneally, subcutaneously, or locally.

77. The method of any of claims 66-76, wherein the FGL2 neutralizing immune cells are administered intravenously, intrathecally, intraventricularly or intracranially.

78. The method of any of claims 66-77, further comprising administering at least a second anticancer therapy to the subject.

79. The method of claim 78, wherein the second anticancer therapy is a surgical therapy, chemotherapy, radiation therapy, cryotherapy, hormonal therapy, immunotherapy or cytokine therapy.

80. The method of claim 79, wherein the second anticancer therapy is chemotherapy.

81. The method of claim 80, wherein the chemotherapy is doxorubicin or cyclophosphamide.

82. The method of claim 78, wherein the second anticancer therapy is radiation therapy.

83. The method of claim 78, wherein the second anticancer therapy comprises a CAR therapy.

84. The method of claim 83, wherein the CAR therapy is CAR T cell therapy.

85. The method of claim 78, wherein the second anticancer therapy comprises an immunomodulator.

86. The method of claim 85, wherein the immunomodulator is a STAT3 inhibitor, an A2AR inhibitor, or an immune checkpoint inhibitor.

87. The method of claim 85, wherein the immunomodulator is a STAT3 inhibitor.

88. The method of claim 85, wherein the immunomodulator is an A2AR inhibitor.

89. The method of claim 85, wherein the immunomodulatory is an immune checkpoint inhibitor.

90. The method of claim 89, wherein the immune checkpoint inhibitor is an anti-CTLA-4 antibody, an anti-PD-L1 antibody, and/or an anti-PD1 antibody.

91. The method of claim 90, wherein the anti-PD1 antibody is nivolumab, pembrolizumab, CT-011, BMS 936559, MPDL328OA or AMP-224.

92. The method of claim 87, wherein the STAT3 inhibitor is WP1066, S3I-201, fludarabine, TTI-101, AZD9150, COPB-31121, OPB-51602, static, niclosamine, nifuroxazide, AS1517499, C188-9, SH-4-54, napabucasin, artesunate, BP-1-1-102, cryototanshinone, SH5-07, ochromycinone, HJC0152, APTSTAT3-9R, or HO-3867.

93. The method of claim 88, wherein the A2AR inhibitor is SCH58261, SYN115, ZM241365, or FSPTP.

94. The method of claim 78, wherein the second anticancer therapy comprises T cell therapy, NK cell therapy, dendritic cell therapy, or a tumor vaccine.

95. The method of claim 78, wherein the FGL2 neutralizing immune cells have enhanced anti-tumor activity as compared to direct FGL2 antibody administration.
